(19) United States
(12) Patent Application Publication
(10) Pub. No.: US 2021/0023248 A1
(43) Pub. Date: Jan. 28, 2021

(54) DISINFECTING APPARATUS

(71) Applicants: James Townsend, Cortland, OH (US); Richard Glen Fletcher, Salem, OH (US)

(72) Inventors: James Townsend, Cortland, OH (US); Richard Glen Fletcher, Salem, OH (US)

(21) Appl. No.: 16/939,508

(22) Filed: Jul. 27, 2020

Related U.S. Application Data
(60) Provisional application No. 62/879,050, filed on Jul. 26, 2019.

(51) Int. Cl. A61L 2/10

(52) U.S. Cl. CPC A61L 2/10; A61L 2202/122; A61L 2202/121; A61L 2202/11; A61L 2202/24

(57) ABSTRACT

An apparatus uses disinfecting ultraviolet light in the C spectrum (UVC) within a housing having an interior chamber. The exterior surfaces of the apparatus are provided in stainless steel suitable for a health care environment. The housing is configured to receive a variety of large devices such as walkers and wheel chairs. Once placed in the interior chamber, first and second offset sanitizing lights are turned on to sanitize the item or items disposed in the interior chamber. The offset lights provide light coverage to both ends of an item such as a wheel chair placed in the interior chamber. The interior of the chamber is lined with reflective, noduled material, to reflect light in a scattered pattern to increase coverage for the item being sanitized. A door closes the interior chamber when the lights are turned on. An interlock is used with the door to turn the lights off when the door is open.

### CROSS REFERENCE TO RELATED APPLICATIONS

This application claims the benefit of U.S. provisional patent application No. 62/879,050 filed Jul. 26, 2019; the disclosures of which are incorporated herein by reference.

### BACKGROUND OF THE DISCLOSURE

### 1. Technical Field

The disclosure generally relates to apparatus used to disinfect items and, more particularly, to disinfecting enclosures that use ultraviolet light to disinfect items placed within the enclosure.

### 2. Background Information

The coronavirus pandemic of 2020 has increased the demand for devices that can thoroughly disinfect various items of a virus. Ultraviolet light having a wavelength in the range of 200 nm to 280 nm is known as UVC light. UVC light inactivates the DNA and RNA of bacteria and viruses, rendering them harmless. UVC light is believed to be an effective disinfecting solution for the novel coronavirus of 2020. For UVC light to effectively work, all of the potentially-contaminated surfaces of an item being disinfected have to be exposed to the light waves for a period of time sufficient to inactive or the virus. At the same time, the disinfecting process must be performed while maintaining the safety of humans using and in proximity to the disinfecting device UVC light can damage human skin and the human eye. As such, the disinfecting process must be shielded from potential human exposure.

The issue of disinfecting large equipment is found in a variety of locations with the users of wheelchairs, patient beds, walkers, tables, carts, and other shared medical equipment. For example, wheelchairs are often used by multiple people and can collect germs (viruses and bacteria) from a variety of sources. Patient beds and carts used with medical testing equipment also can collect germs and need to be disinfected. Other smaller equipment such as blood pressure cuffs and stethoscopes also can be disinfected after each use. Such disinfection should lead to lower infection rates.

### SUMMARY OF THE DISCLOSURE

In one configuration, the disclosure provides a disinfecting apparatus and method that uses ultraviolet light within a closed housing having an interior chamber. When items to be disinfected such as wheel chairs, walkers, or carts are moved into the interior chamber, a door must be closed before the ultraviolet disinfecting lamps are turned on to bathe the article in UVC spectrum wavelength light. A door interlock system prevents the light from being turned on while the door is open.

The interior chamber of the disinfecting apparatus is defined by interior walls that are reflective and include irregularities such as nodules to reflect the light from the UVC lamps. The reflection increases the efficiency of the disinfecting system. A mirror type surface enhances the reach of the UVC light rays on the item disposed in the housing. One exemplary material that may be used is a bright-finished nodular or polished aluminum tread or diamond plate. Another example is a metalized polymer material such as metalized polyethylene or metalized corrugated material. Both of these materials may be formed to have surface irregularities.

The disclosure provides a plurality of light sources that are offset with respect to each other to increase the coverage inside of the enclosure. The light sources are disposed in a cavity disposed above the level of the top of the door to minimize the likelihood they will be struck by a device being placed into or removed from the enclosure. In other configurations, the plurality of all or some of the light sources can be disposed along the walls and door. The lights are Teflon® coated to provide shatter resistant bulbs.

The disclosure provides an apparatus that is powered by a standard 120 Volt source of electricity. The apparatus can be readily moved to different locations where it can be plugged in, used, and then moved again because it does not need to be hardwired or plugged into a 220 Volt power source.

The disclosure provides a system wherein the apparatus must be unlocked by a registered user prior to use. The registered user inputs a security code or scans their user identification which allows the apparatus to be used. The system then allows the user to identify the item being disinfected by scanning a bar code carried by the item. The system logs the item, the time, and date of the scan. The system then identifies the length of exposure for that item and controls the lights after the item is placed into the enclosure and the door is closed. The system includes an output port such as a USB port that allows information in the computer's memory to be off loaded. The computer also can be accessed through a wireless connection to allow its data to be accessed by another computer. The system includes a counter which tells the user how many hours the lamps have been in use and when they are recommended to be changed.

In one configuration, the apparatus includes a frame covered with stainless steel on the outside and nodular plate on the inside. Another configuration uses plastic for the frame or outer panels.

In one configuration, the apparatus includes a storable ramp that provides for easy entry and exit from the chamber for wheeled items or items that are slid along the floor.

The disclosure provides an apparatus with an emergency stop switch. The apparatus also includes a lock-out switch that prevents accidental operation during service.

The system is equipped with a shut down mode should the door be opened during a disinfecting cycle. If the door is opened, the cycle immediately stops and the display screen will display an incomplete cycle warning which requires the user to perform a complete restart of cycle.

In one configuration, the apparatus includes a removable front panel installed for easy access to the electrical connections.

Some configurations of the apparatus are mobile and include self-contained casters and levelers.

The preceding non-limiting aspects, as well as others, are more particularly described below. A more complete understanding of the apparatus and methods can be obtained by reference to the accompanying drawings, which are not intended to indicate relative size and dimensions of the assemblies or components thereof. In those drawings and the description below, like numeric designations refer to components of like function. Specific terms used in that description are intended to refer only to the particular structure of the embodiments selected for illustration in the drawings, and are not intended to define or limit the scope of the disclosure.

### BRIEF DESCRIPTION OF THE DRAWINGS

FIG. 1 is a perspective view of an exemplary disinfecting apparatus with its door open and the light units depicted in dashed lines.

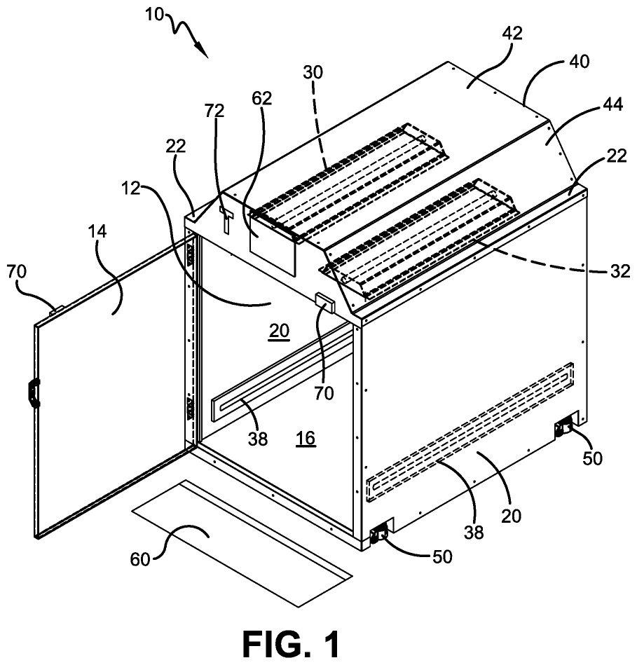

FIG. 2 is a front view of the exemplary disinfecting apparatus with the door closed and the protective panel removed to show the control panel.

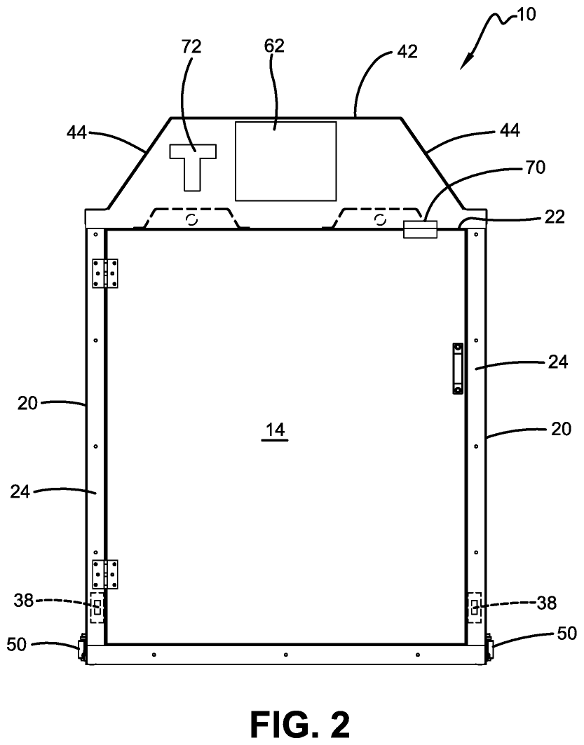

FIG. 3 is a top plan view of FIG. 2.

FIG. 4 is a right side view of FIG. 2.

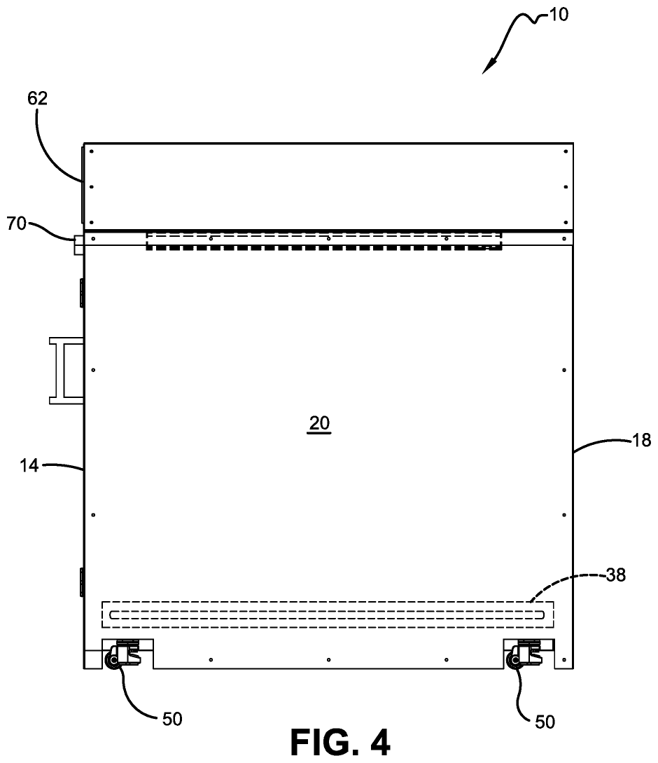

### DETAILED DESCRIPTION OF THE DISCLOSURE

An exemplary configuration of a disinfecting apparatus is indicated generally by the reference numeral 10 in the accompanying drawings. Apparatus 10 disinfects items placed in its interior chamber 12 with germicidal UVC wavelength radiation. UVC is ultraviolet light in the C spectrum (UV-C) is energy-rich light with a wavelength of 200-400 nanometers (nm) with wavelengths from 200 nm to 280 nm being effective for disinfecting surfaces. Apparatus 10 provides a complete enclosure for the item being disinfected with no light leakage during its operation. The exterior of apparatus 10 is provided in stainless steel suitable for use in health care facilities.

Apparatus 10 includes a plurality of walls that define interior chamber 12. A door 14 that forms part of a wall or, alternatively, that takes the place of one of the walls, provides selective access to interior chamber 12 when door 14 is in an open condition. Door 14 is connected with hinges that allow door 14 to be lifted off of apparatus for cleaning. In the exemplary configuration, interior chamber 12 is defined by a floor 16, a rear wall 18, a pair of side walls 20, a ceiling 22, and a front wall 24 that includes the interior surface of door 14 when door 14 is closed. At least the interior-facing surfaces of floor 16, rear wall 18, side walls 20, and front wall 24 and/or door 14 are reflective and can be polished aluminum that reflects similar to a mirror. In the exemplary configuration, at least these interior surfaces are irregular to help reflect the light in a scattered pattern. The surfaces can include irregular nodules or a series of raised areas such as provided by bright-finished nodular or polished aluminum tread or diamond plate. In the exemplary configuration, all six interior surfaces, including floor 16 and ceiling 22, are reflective and irregular.

In one configuration, apparatus 10 has double-panel walls such that at least its rear wall 18, side walls 20, and front wall 24 and door 14 have exterior stainless steel panels and interior reflective irregular aluminum panels spaced from the exterior panels to define an air gap between the exterior and interior panels. The panels are carried on an aluminum frame. The stainless steel panels are 304 polished stainless steel with a #4 finish. The interior panels are polished aluminum tread or polished diamond plate aluminum. The reflective interior surfaces reflect the UVC light waves and increase the efficiency of the disinfecting system. A mirror type surface enhances UV light rays which, in turn greatly increases the effectiveness of the UVC device on pathogens and viruses. It is also believed imperfections or irregularities further increase the effectiveness of the UVC disinfecting device.

In the exemplary configuration depicted in the drawings, the interior chamber is sized for a variety of equipment such as wheel chairs, walkers, equipment racks, wheeled tables, and the like. When apparatus 10 is configured for stretchers, cots, and beds, apparatus 10 is lengthened to have a depth sufficient to receive the longer items. When used with wheeled beds, bumper rails are disposed along both side walls 20 and rear wall 18 to help guide the bed into chamber 12 and to protect the interior of apparatus 10. In an exemplary configuration for a bed, interior chamber 12 can have a depth of eight feet.

First 30 and second 32 double-offset disinfecting germicidal UVC lamps are disposed above ceiling 22 and direct their light down through openings defined by ceiling 22 into interior chamber 12. Each lamp is a 36 inch UVC disinfecting lamp. Both lamps can be UVC LED. The user places an item to be disinfected into interior chamber 12, closes door 14, and then turns first 30 and second 32 lamps on for a selected time period to provide the disinfecting function. The UVC rays inactivate microorganisms by disrupting their cellular membranes and by damaging their DNA or RNA. The UVC rays initiate a reaction between two molecules of thymine, one of the bases that make up DNA. Light rays at 254 nm wavelength (germicidal UV or UVC) causes adjacent thymine molecules on DNA to dimerize. The thymine dimer is very stable. When enough of these defects accumulates on a microorganism DNA its replication is inhibited, results in effective UV disinfection by rendering the microorganisms harmless. The germicidal ultraviolet (UVC) light is in the region of maximum germicidal effectiveness and is highly lethal to virus, bacteria, protozoa and mold.

The non-ozone producing, UVC bulbs remain 100% effective for 12,000 hours or two years, whichever occurs first. After two years the effectiveness of the bulb begins to decline. In humans, UVC is absorbed in the outer dead layers of the epidermis. Accidental overexposure to UVC can cause corneal burns, commonly termed welders' flash, and snow blindness, a severe sunburn to the face. While UVC injury usually clears up in a day or two, it can be painful. The device configurations described herein are thus designed to prevent user exposure to the light from the UVC lamps.

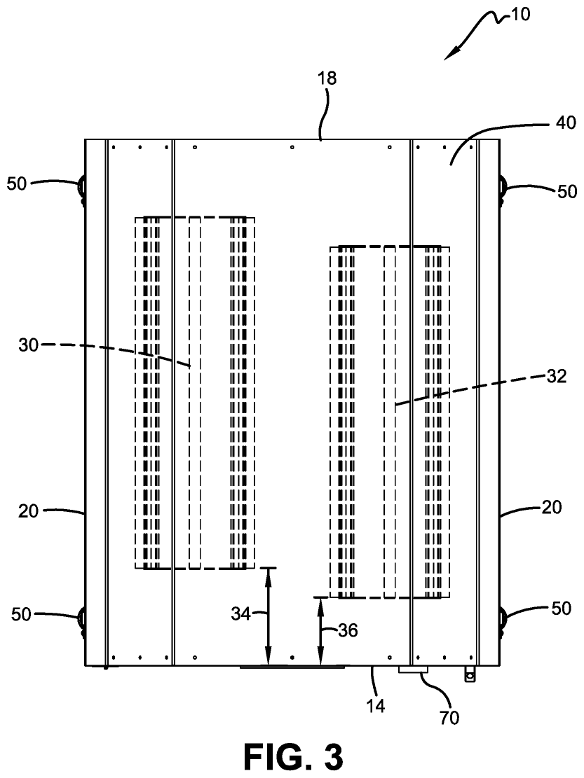

Double-offset lamps 30 and 32 are offset side to side and back to front to provide coverage for items in apparatus 10. Lamps 30 and 32 are proprietary to apparatus 10 to ensure only the correct lamps are installed. A single ballast is provided for both lamps 30 and 32. In the exemplary configuration, each lamp 30 and 32 is closer to a sidewall than the other lamp. As shown in FIG. 3, lamp 30 is disposed at a location that is ten to thirty-three percent of the width of apparatus from the left side of apparatus. Lamp 32 is disposed at a location that is ten to thirty-three percent of the width of apparatus from the left side of apparatus. In one configuration, a location that is twenty to twenty-five percent of the width provides good coverage of the sides of items placed in interior chamber 12. This location offsets lamps 30 and 32 laterally so there is more space between lamps 30 and 32 than between either lamp and a side wall 20.

Lamps 30 and 32 are also offset front to back such that the distance (reference numeral 34) between the front end of lamp 30 to the interior of door 14 is different than the distance (reference numeral 36) between the front end of lamp 32 and interior of door 14. The front end of lamp 30 is at least three times closer to or farther from front wall 24 than lamp 32. If the front end of lamp 30 is spaced four inches from the interior of front wall 24, then the front end of lamp 32 is at least twelve inches from the interior of front wall 24. In longer configurations of apparatus 10 used for beds, additional lamps are used and are aligned rearwardly with lamps 30 and 32.

An alternative configuration includes additional lamps 38 carried by the lower thirds of side walls 20 to provide light to the lower portions and undersides of items placed in apparatus 10. An exemplary position is twenty-five percent of the height of side wall 20. These lamps are disposed in recesses defined between the inner and outer surfaces of side walls 20 so there is little risk of items impacting them as they are moved into and out of chamber 12. Transparent covers are used to protect the lamps.

Lamps 30 and 32 as well as the controls for operating apparatus are carried by or disposed within a roof enclosure 40. Roof enclosure 40 includes a flat upper roof 42 and angled risers 44 that extend down from the lateral edges of flat upper roof 42 to a location spaced inwardly of the tops of sidewalls 20. Lamps 30 and 32 are disposed within roof enclosure 40 above ceiling 22. The front panel of roof enclosure is removable to provide access to the ballast for lamps 30 and 32 as well as the electrical connections for the other elements of apparatus 10.

In some configurations, apparatus 10 includes wheels 50. Wheels 50 are threadedly supported so they can be extended to lift apparatus off the ground onto wheels 50 or retracted to allow apparatus to rest on the floor. Wheels 50 can be locked and used to level apparatus 10.

Roof enclosure 40 includes a reflective lamp housing for each lamp 30 and 32. The interior of lamp housing is polished to reflect light into interior chamber 12 through openings defined by ceiling 22. Each lamp housing can be continuously curved (such as a semi-circle) or have multiple angled walls such as the three depicted in the drawings. These housings position each lamp 30 and 32 above ceiling 22 where they are protected from people and items moving in and out of apparatus 10. Each lamp 30 and 32 can be surrounded by a safety enclosure to protect the bulbs and trap broken glass in the case of a broken bulb. Alternatively, each lamp 30 and 32 can be coated with a polymer coating to limit the risk of shattering.

A ramp 60 is carried between installed and storage positions at the bottom portion of front wall 24. Ramp 60 can be lifted and slid into chamber 12 for storage. When apparatus is to be used, ramp 60 is pulled out wherein its rear edge engages and locks to connectors disposed along front wall 24 under door 14. Ramp 60 allows wheeled items to be rolled into enclosure 12. Door 14 fully closes when ramp 60 is in the installed condition. Ramp 60 can be removed and stored within enclosure 12 when apparatus is being moved or when it is not in use. Alternatively, ramp 60 can be hinged and folded inwardly to a storage condition.

Apparatus is powered by standard 120 Volt electrical power and includes a cord and plug that allow it to be powered by a standard electrical outlet. Apparatus 10 can be run manually or through a program run by computer 62. Computer 62, which can be in the form of a programmable logic controller, controls the operation of apparatus 10. Computer 62 can be used to turn lamps 30 and 32 on and off, to prevent lamps 30 and 32 from being turned on if the door is open, to control how long lamps 30 and 32 remain on during a disinfecting process, and to maintain a log of the disinfecting runs which can be supplemented with the identification of the items being disinfected through bar code numbers or a manual identification of the items by the user. Computer 62 includes an input device in the form of a key board or a touch screen which also functions to display information about apparatus 10.

In the manual configuration, apparatus includes a switch that allows a user to turn lamps 30 and 32 on and off. This configuration either allows the user to define the amount of time for the disinfection process or a timer is used to automatically turn lamps 30 and 32 off after a set period of time. The activation switch is either a mechanical switch or is provided on a touch screen input for computer 62. In an exemplary manual configuration, lamps 30 and 32 are turned on for thirty seconds after door 14 is closed and the switch is pressed to the cony condition. The countdown can be displayed on the screen. Apparatus 10 includes a door interlock system that includes a switch 70 that can be a magnetic switch. Apparatus will not turn lamps 30 and 32 to the cony condition unless switch 70 is in the closed condition. If switch 70 is moved to an open condition when lamps 30 and 32 are on, the controller immediately turns lamps 30 and 32 off and displays a warning that the disinfection process was incomplete.

Apparatus 10 also includes a lockout/tagout power switch that allows a worker to put a lock on the switch to prevent apparatus from being powered when the lock is on the switch. Apparatus also includes an emergency power disconnect switch is also provided to cut power to apparatus 10.

In another configuration, controller 62 locks the operation of apparatus 10 until security information is entered. The security information can be a password provided through the touch screen or information provided by scanning a security badge, fingerprint, or facial recognition. This configuration prevents lamps 30 and 32 from being turned on until an authorized user has logged into computer 62 and provided security access information. Only those trained in the use of apparatus 10 would be provided with this access. Once a user has properly accessed apparatus 10, the user starts a disinfecting cycle by loading the items to be disinfected into enclosure 12, closing door 14, and selecting a start option. When the start option is selected, computer 62 checks for the closed status of door 14 through interlock 70 and then powered lamps 30 and 32 for a set amount of time, such as thirty seconds to a minute, and then turns lamps 30 and 32 off. Once the lamps are off, the user opens door 14 and remove the disinfected items. If door 14 is opened while lamps 30 and 32 are on, the interlock is triggered and computer 62 turns lamps 30 and 32 off and an alarm may sound or an alarm message is displayed on the screen. The user would have to run the process again. Interlock 70 does not prevent door 14 from being opened during the disinfecting process as a safety measure.

Another configuration of apparatus 10 includes a bar code reader 72 that allows a user to log bar coded items being disinfected into the log of computer 62. The operator of apparatus 10 can keep records of the date time, and duration of disinfection.

In the foregoing description, certain terms have been used for brevity, clearness, and understanding. No unnecessary limitations are to be implied therefrom beyond the requirement of the prior art because such terms are used for descriptive purposes and are intended to be broadly construed. Moreover, the description and illustration of the invention is an example and the invention is not limited to the exact details shown or described. Modifications and alterations of those embodiments will be apparent to one who reads and understands this general description. The present disclosure should be construed as including all such modifications and alterations insofar as they come within the scope of the appended claims or equivalents thereof. Throughout the description and claims of this specification the words “comprise” and “include” as well as variations of those words, such as “comprises,” “includes,” “comprising,” and “including” are not intended to exclude additives, components, integers, or steps.

1. An apparatus for disinfecting an item; the apparatus comprising:
a housing defining an interior chamber; the housing having an opening that allows the item to be sanitized to be placed into the interior chamber of the housing;
the chamber being partially defined by first and second side walls and a ceiling;
first and second UVC lamps disposed above the ceiling; the first and second lamps selectively providing UVC light to the interior chamber of the housing;
the first lamp being spaced from the first side wall by a first distance;
the second lamp being spaced from the second side wall by a second distance;
the first limo being spaced from the second lamp by a third distance; and
each of the first and second distances being less than the third distance.

2. The apparatus of claim 1, wherein the housing has an interior surface; the interior surface being a reflective and irregular surface.

3. The apparatus of claim 2, wherein the reflective and irregular surface interior surface of the housing is a surface of a section of polished aluminum tread.

4. The apparatus of claim 2, wherein the reflective and irregular surface interior surface of the housing is a surface of a section of polished diamond plate.

5. The apparatus of claim 2, wherein the reflective and irregular surface interior surface of the housing is a surface of a section metalized polymer material.

6. The apparatus of claim 2 wherein the reflective and irregular surface interior surface of the housing is a surface of a section metalized corrugated material.

7. The apparatus of claim 1, wherein each of the first and second UVC lamps includes a protective coating.

8. The apparatus of claim 7, wherein the protective coating is shatter-proof.

9. The apparatus of claim 1, further comprising a door having open and closed conditions; the open condition of the door allowing access to the interior chamber; the closed condition of the door limiting access to the interior chamber;
a switch connected to the first and second lamps;
the switch turning the first and second lamps to an off condition when the door is in the open condition.

10. The apparatus of claim 1, further comprising a ramp movable between stored and installed positions.

11. The apparatus of claim 1, wherein the chamber is partially defined by a front wall;
the first lamp being spaced from the front wall by a fourth distance;
the second lamp being spaced from the front wall by a fifth distance; and
the fourth distance being different than the fifth distance.

12. The apparatus of claim 1, further comprising a third UVC lamp carried by the first side wall and a fourth UVC lamp carried by the second side wall.

13. The apparatus of claim 12, wherein each of the first and second side walls has a height; the third lamp being disposed at a height that is less than one third of the height of the first side wall; and the fourth lamp being disposed at a height that is less than one third of the height of the second side wall.

14. The apparatus of claim 12, wherein the third lamp is disposed within a cavity defined by the first side wall and the further lamp is disposed within a cavity defined by the second side wall.

15. An apparatus for disinfecting an item; the apparatus comprising:
a housing defining an interior chamber; the housing having an opening that allows the item to be sanitized to be placed into the interior chamber of the housing;
the chamber being partially defined by first and second side walls, a front wall, and a ceiling;
first and second UVC lamps disposed above the ceiling; the first and second lamps selectively providing UVC light to the interior chamber of the housing;
the first lamp being spaced from the front wall by a first distance;
the second lamp being spaced from the front wall by a second distance; and
the first distance being different than the second distance.

16. An apparatus for disinfecting an item; the apparatus comprising:
a housing defining an interior chamber; the housing having an opening that allows the item to be sanitized to be placed into the interior chamber of the housing;
the chamber being partially defined by first and second side walls and a ceiling;
first and second UVC lamps disposed above the ceiling; the first and second lamps selectively providing UVC light to the interior chamber of the housing;
a third UVC lamp carried by the first side wall; and
a fourth UVC lamp carried by the second side wall.

17. The apparatus of claim 16, wherein each of the first and second side walls has a height; the third lamp being disposed at a height that is less than one third of the height of the first side wall; and the fourth lamp being disposed at a height that is less than one third of the height of the second side wall.

18. The apparatus of claim 16, wherein the third lamp is disposed within a cavity defined by the first side wall and the further lamp is disposed within a cavity defined by the second side wall.

19. The apparatus of claim 15, further comprising a transparent cover for each side wall lamps.
